(19) United States
(12) Patent Application Publication
(10) Pub. No.: US 2018/0193620 A1
(43) Pub. Date: Jul. 12, 2018

(54) LUBRICANT DISPENSING DEVICE

(71) Applicant: Maxine Lynn Barasch, Albany, NY (US)

(72) Inventor: Maxine Lynn Barasch, Albany, NY (US)

(21) Appl. No.: 15/867,179

(22) Filed: Jan. 10, 2018

Related U.S. Application Data
(60) Provisional application No. 62/444,568, filed on Jan. 10, 2017.

(51) Int. Cl. A61M 31/00; A61H 19/00

(52) U.S. Cl. CPC A61M 31/00; A61H 19/32; A61H 19/50; A61H 2205/087; A61M 2205/3592; A61M 2205/8206; A61M 2205/3344; A61M 2205/3368; A61M 2205/36

(57) ABSTRACT

Embodiments of the invention provide a lubricant dispensing device, which includes a ring, at least one hollow chamber, and at least one dispenser. The ring is sized and configured for attaching to a body part, typically, a penis of a user (though other configurations are possible). The at least one hollow chamber is configured to hold lubricant or other suitable substance. The lubricant dispenser is configured to squirt out lubricant when actuated by a user. In use, the device provides convenient lubrication during intercourse or other sexual activity. The device may include a heating element to heat the lubricant prior to dispensing. The device may further include a vibrator to impart massage to the user, or to the user's sexual partner during sexual activity.

### CROSS-REFERENCE TO RELATED APPLICATION

The present patent document claims priority to, U.S. Provisional Patent Application No. 62/444,568 filed Jan. 10, 2017, the entire contents of which is incorporated herein by reference.

### FIELD OF THE INVENTION

Embodiments of the invention relate to lubricant dispensing devices, and more particularly, to rings which dispense lubricant.

### BACKGROUND

During sex with a partner, or masturbation with a dildo, a woman's body has glands that release lubrication into the vagina. For some women, the vagina does not naturally, on its own, become lubricated enough for comfortable sex. This can be due any of a number of factors, such as arousal level, physiological characteristics, etc. In addition, for anal sex, artificial lubrication is typically required for comfort and safety. Using bottled lubricants during sex requires sex partners to interrupt intercourse to find a bottle of lubricant, squirt it where necessary, and then begin intercourse again. This is cumbersome, and interruptive. There exists a need for improvements in lubricant dispensing.

### SUMMARY

Embodiments of the invention provide a lubricant dispensing device, which includes a ring, at least one hollow chamber, and at least one dispenser. The ring is sized and configured for attaching to a body part, typically, a penis of a user (though other configurations are possible). The at least one hollow chamber is configured to hold lubricant or other suitable substance. The lubricant dispenser is configured to squirt out lubricant when actuated by a user. In use, the device provides convenient lubrication during intercourse or other sexual activity. The device may include a heating element to heat the lubricant prior to dispensing. The device may further include a vibrator to impart massage to the user, or to the user's sexual partner during sexual activity.

### BRIEF DESCRIPTION OF THE DRAWINGS

The accompanying drawings, which are incorporated in and constitute a part of this specification, illustrate several embodiments of the present teachings and together with the description, serve to explain the principles of the present teachings.

FIG. 1 shows a front view of a lubricant dispensing device in accordance with some embodiments of the invention.

FIG. 2A shows a front view of lubricant dispensing device in accordance with some embodiments of the invention.

FIG. 2B shows a side view of the lubricant dispensing device of FIG. 2A.

FIG. 2C shows the back of such lubricant dispensing device of FIG. 2A.

FIG. 3 shows a perspective view of lubricant dispensing device in accordance with some embodiments of the invention.

FIG. 4 shows a perspective view of lubricant dispensing device in accordance with some embodiments of the present invention.

FIG. 5 shows a front view of lubricant dispensing device in accordance with some embodiments of the invention.

FIG. 6 shows a front view of lubricant dispensing device in accordance with some embodiments of the invention.

FIG. 7 shows a lubricant dispensing device in accordance with some embodiments of the invention.

FIG. 8 shows a front view of lubricant dispensing device in accordance with some embodiments of the invention.

FIG. 9 shows a lubricant dispensing device in accordance with some embodiments of the invention.

FIG. 10A shows a front view of lubricant dispensing device with a channel in accordance with some embodiments of the invention.

FIG. 10B shows a lubricant dispensing device with another channel in accordance with some embodiments of the invention.

FIG. 10C shows a lubricant dispensing device with yet another channel in accordance with some embodiments of the invention.

FIG. 11A shows a side view of lubricant dispensing device in accordance with some embodiments of the invention.

FIG. 11B shows a front view of the lubricant dispensing device of FIG. 11A.

FIG. 11C shows a top-down view of the lubricant dispensing device of FIG. 11A.

FIG. 12 shows a side view of a lubricant dispensing device in accordance with some embodiments of the invention.

FIG. 13A shows a side view of a lubricant dispensing device having a pump dispenser in accordance with some embodiments of the invention.

FIG. 13B shows a front view of the lubricant dispensing device of FIG. 13A.

FIG. 13C shows a top view of the lubricant dispensing device of FIG. 13A.

FIG. 14 shows a side view of a lubricant dispensing device having a heating element in accordance with some embodiments of the present invention.

FIG. 15A shows a side view of a button operated lubricant dispensing device in non-dispensing configuration in accordance with some embodiments of the invention.

FIG. 15B shows a front view of the plunger with the via of FIG. 15A.

FIG. 15C shows lubricant dispensing device of FIG. 15A except in a dispensing configuration.

FIG. 15D shows a front view of lubricant dispensing device of FIG. 15C in dispensing configuration.

FIG. 15E shows a top-down view of the lubricant dispensing device of FIG. 15A.

FIG. 16 shows a block diagram in accordance with some embodiments of the invention.

The drawings are not necessarily to scale. The drawings are merely representations, not intended to portray specific parameters of the invention. The drawings are intended to depict only typical embodiments of the invention, and therefore should not be considered as limiting in scope. In the drawings, like numbering represents like elements. Furthermore, certain elements in some of the figures may be omitted, or illustrated not-to-scale, for illustrative clarity. Typically, dotted or dashed lines in the figures indicate that the element is below or being covered by another element.

### DETAILED DESCRIPTION

Embodiments of the invention provide a lubricant dispensing device, which includes a ring, at least one hollow chamber, and at least one dispenser. The ring is sized and configured for attaching to a body part, typically, a penis of a user (though other configurations are possible). The at least one hollow chamber is configured to hold lubricant or other suitable substance. The lubricant dispenser is configured to squirt out lubricant when actuated by a user. In use, the device provides convenient lubrication during intercourse or other sexual activity. The device may include a heating element to heat the lubricant prior to dispensing. The device may further include a vibrator to impart massage to the user, or to the user's sexual partner during sexual activity.

Reference throughout this specification to “one embodiment,” “an embodiment,” “some embodiments”, or similar language means that a particular feature, structure, or characteristic described in connection with the embodiment is included in at least one embodiment of the present invention. Thus, appearances of the phrases “in one embodiment,” “in an embodiment,” “in some embodiments”, and similar language throughout this specification may, but do not necessarily, all refer to the same embodiment.

Moreover, the described features, structures, or characteristics of the invention may be combined in any suitable manner in one or more embodiments. Aspects described with respect to one embodiment may be combined with other embodiments. Features of the embodiments may be “mixed and matched” in any suitable way. It will be apparent to those skilled in the art that various modifications and variations can be made to the present invention without departing from the spirit and scope and purpose of the invention. Thus, it is intended that the present invention cover the modifications and variations of this invention provided they come within the scope of the appended claims and their equivalents. Reference will now be made in detail to the preferred embodiments of the invention.

The terminology used herein is for the purpose of describing particular embodiments only and is not intended to be limiting of this disclosure. As used herein, the singular forms “a”, “an”, and “the” are intended to include the plural forms as well, unless the context clearly indicates otherwise. Furthermore, the use of the terms “a”, “an”, etc., do not denote a limitation of quantity, but rather denote the presence of at least one of the referenced items. The term “set” is intended to mean a quantity of at least one. It will be further understood that the terms “comprises” and/or “comprising”, or “includes” and/or “including”, or “has” and/or “having”, when used in this specification, specify the presence of stated features, regions, integers, steps, operations, elements, and/or components, but do not preclude the presence or addition of one or more other features, regions, integers, steps, operations, elements, components, and/or groups thereof.

For the purpose of ease of description, the examples describe the lubricant-dispensing ring positioned on a penis. It may be positioned on the penis shaft in front of the scrotum, or behind the scrotum. However, the ring could instead be installed on a dildo, vibrator, butt plug, or other device. Alternatively, the ring could be worn on a body part other than the penis, such as, without limitation, a wrist or finger. For example, the ring could be configured as a bracelet to be worn around the wrist of a user instead of a penis.

For the purposes of disclosure, the term, “substantially” is defined as “for the most part”. It means “to a great extent,” but having some room for some minor variation.

For the purposes of disclosure, the terms “top,” “bottom,” “side,” “upward,” “downwards,” and similar terms are used for ease of description assuming the lubricant dispensing device is positioned as shown in the Figures. These words are not meant to be limiting, as the lubricant dispensing device may be positioned differently where desired by a user and where feasible.

For the purposes of disclosure, the term “lubricant” is defined as fluid, liquid, gel, silicon, jelly, oil, lotion, aqueous solution, water, or other suitable flowable material. In embodiments, the lubricant causes a surface including a user's body part, sex toy, or other surface to be “slippery” enabling sliding for a more comfortable and pleasurable experience during sexual intercourse, massage, etc.

For the purposes of disclosure, the term “seal” is meant to mean a closure (or substantial closure) when used as a noun, and to close (or to substantially close) when used as a verb.

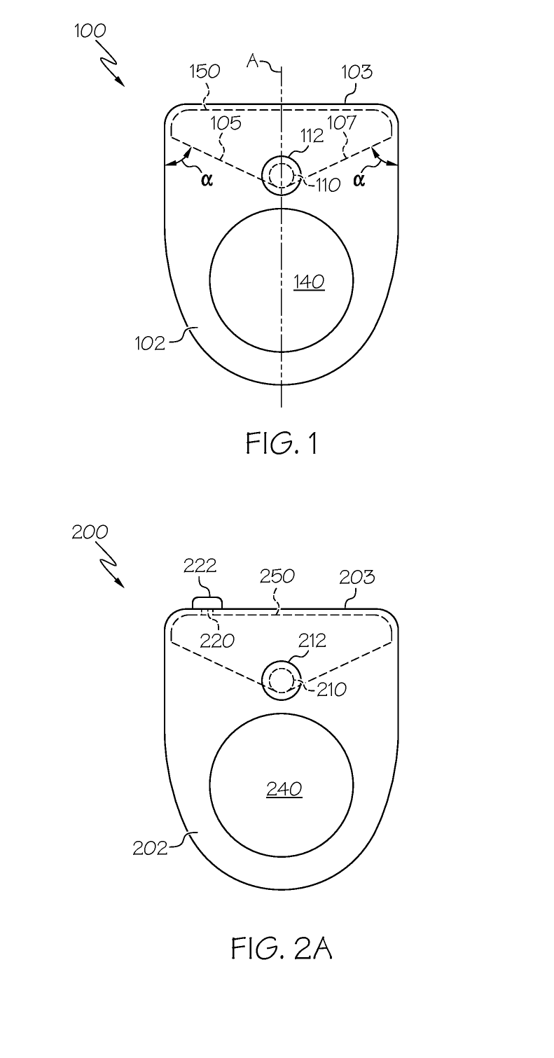

FIG. 1 shows a front view of lubricant dispensing device 100 in accordance with some embodiments of the invention. Penis ring 100 has a ring 102 and a hollow chamber 150 surrounded by housing 103. A user can insert his penis through an insert hole 140 outlined by the ring 102. In some embodiments, the ring 102 is flexible and resilient such that it is sized and configured to tightly grip a penis during use. This grip may be tight enough to slow down blood flow into the penis, extending the time of the user's erection prior to orgasm. The chamber includes a first sealable opening 110 configured to open to receive lubricant into the chamber and dispense lubricant from the chamber. First sealable opening has first seal 112 which serves to open and close the opening. As shown, the hollow chamber 150 is situated above the ring. It should be recognized that, in some embodiments, the hollow chamber 150 can be situated in other orientations, and all such orientations are included within the scope and spirit of the invention.

The “first sealable opening” and the “first seal” together are a “dispenser” though in some embodiments, such dispenser may also be the recipient opening for lubrication. Note that in the examples herein, the first seal and first sealable opening can be any suitable type of dispenser that will allow lubricant out of the chamber, while also sealing it shut when needed. Accordingly, while in some of the examples, a seal and a sealable opening are shown with separate lead lines, that is for ease of discussion. In some embodiments, the seal and sealable opening can be a single complex. Non-limiting examples are described herein. In some embodiments, the first sealable opening is also configured to receive lubricant therein as well to stock the hollow chamber with lubricant to dispense. Also, the terms “first” and “second”, and other numerical indicators, are not meant to be limiting herein. For example, in some embodiments, “dispenser” may be used for the first seal/sealable opening complex and the term “a seal” and “a sealable opening” may be used for another device into which lubricant can be poured to be stored in the hollow chamber.

In some embodiments, the sealable opening 110 is centered horizontally on axis A between the lateral walls of the housing surrounding the hollow chamber. In some embodiments, the sealable opening is positioned just above (e.g., 1-3 mm) the bottom of the chamber 150 such that when the opening is open, the lubricant will flow out, with minimal drip over the outside housing of the chamber. This allows the lubricant to hit the penis without creating a mess on the outer wall of the chamber. Gravity will push the lubricant to the bottom of the chamber.

In some embodiments, the chamber has at least two interior walls 105 and 107, which are each slanted toward the sealable opening, forming a “funnel” effect. This enhances the lubricant's flow in the chamber downward toward the sealable opening when the ring is positioned upright. In some embodiments, the chamber has lower walls that slant such that the lower walls increase in height as they extend substantially from the bottom of the chamber (or sealable opening) to the side walls (and/or top) of the chamber. In some embodiments, the interior walls are at an angle α from the lateral side walls.

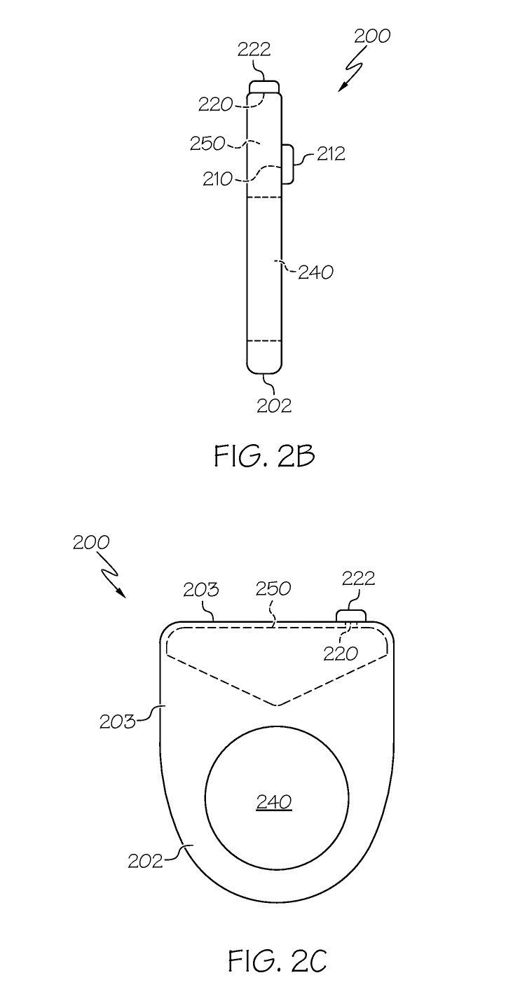

FIG. 2A shows a front view of lubricant dispensing device 200 in accordance with some embodiments of the invention. FIG. 2B shows a side view of such lubricant dispensing device 200. FIG. 2C shows the back of such lubricant dispensing device 200. Lubricant dispensing device 200 has a ring 202 and a hollow chamber 250, both surrounded by housing 203. A user can insert his penis through an insert hole 240 outlined by the ring 202. The chamber includes a first sealable opening 210 configured to open to receive lubricant into the chamber 250. Sealable opening has seal 212 which serves to open and close the opening. The chamber includes a second sealable opening 220 configured to open to dispense lubricant from the chamber 250. Sealable opening has second seal 222 which serves to open and close the opening. Note that in the examples herein, the second seal 222 and second sealable opening 220 can be any suitable type of receiving mechanism that will allow lubricant into the chamber, while also sealing it shut when needed. The apparatuses for opening 220 and sealing 222 can be separate, or be a single device one and the same with each other.

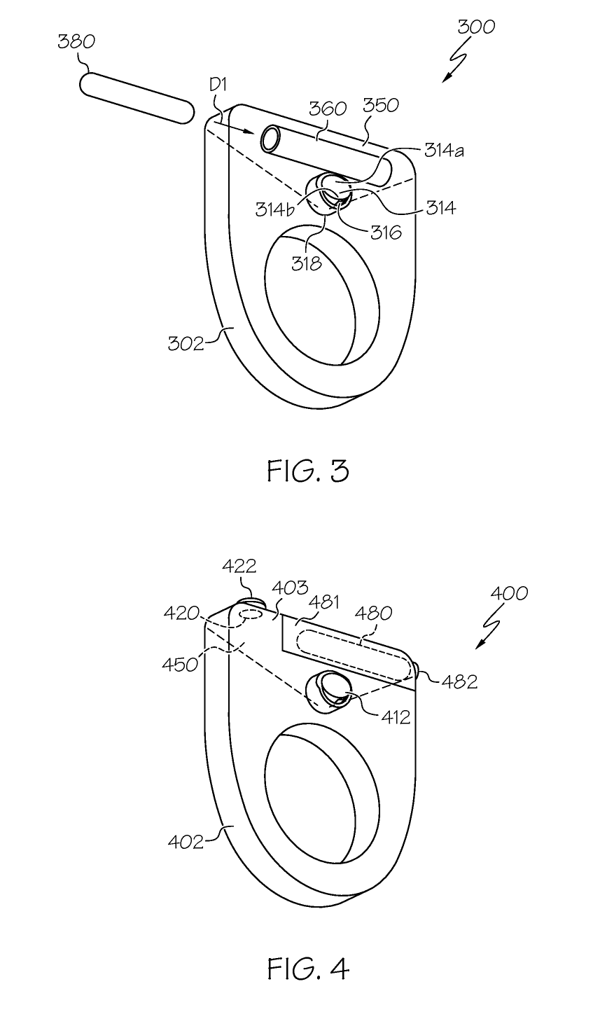

FIG. 3 shows a perspective view of lubricant dispensing device 300 in accordance with some embodiments of the invention. Lubricant dispensing device 300, like lubricant dispensing devices 100 and 200 includes a ring 302 and a hollow chamber 350. It also includes at least one sealable opening 316 and seal 314. In the embodiment, the sealable opening and seal is a disc top dispensing cap. The disc 314 is mounted on a pivot point to cap mount 318. In non-dispensing configuration, the opening 316 is covered by an inner all of mount 318. In dispensing configuration, as shown, when a user presses the top portion 314a of the disc 314 inward (toward the back of the device), the disc pivots backwards, revealing opening 318 on its underside. Lubricant can then flow out of the chamber through opening 316. In some cases, the user can press on the posterior wall of the device to squeeze lubricant out. In such cases, the housing surrounding the hollow chamber 350 are flexible (such as a soft plastic or silicon) such that they can be pushed inward to compress the contents therein. The user can then press the bottom portion 314b of the cap 314 inward to seal the opening 318. Lubricant dispensing device 300 includes a vibrator mount 360 for receiving a removable vibrator. In some embodiments, the vibrator mount 360 may be a sleeve, as shown, which the vibrator 380 slips into by a user pushing through a first end of the sleeve 360 in direction D1. The vibrator 380 may be a “bullet” vibrator, meaning it is a substantially elongated cylinder in shape. The sleeve 360 may comprise a flexible material in order to provide a snug fit around the vibrator 380 to hold the vibrator 380 in place. Although a sleeve is described, the mount may be any mechanism which removably (or in some embodiments, permanently) attaches the vibrator 380 to the lubricant dispensing device 300.

FIG. 4 shows a perspective view of lubricant dispensing device 400 in accordance with some embodiments of the present invention. Lubricant dispensing device 400, like rings 100 and 200 includes a ring 402 and hollow chamber 450. It also includes at least one first sealable opening 410 and first seal 412. In some embodiments, it also includes a second sealable opening 420 and second seal 422. Lubricant dispensing device 400 includes vibrator 480. The vibrator 480 is shown on the interior of the housing of the lubricant dispensing device in a chamber 481 separate from the hollow chamber 450, but in other embodiments, the vibrator may be attached to the exterior housing 403. Button 482 allows a user to power on and off the vibrator 480, as well as scroll through vibration intensity settings (such as high, medium, and low). It should be recognized that the size, shape, and position of the vibrator may be any suitable size, shape, and position, all of which are included within the scope of the invention. In some embodiments, more control buttons are included, or the button 482 is in a different position from that shown. In addition, in some embodiments, 482 may represent a sensor instead of a button, such as a pressure sensor, for actuating the vibrator.

In some embodiments, the vibrator 380 or 480 is an electromechanical vibrator, which may be a geared motor mechanism, which may have, e.g., an asymmetrical load affixed to a rotating shaft, a linear resonant actuator, or a pancake vibration motor, etc. It will be recognized that any suitable vibration or massage mechanism now known or hereafter developed may be substituted for, or used in addition to, an electromechanical vibrator. For example, pulsation (from a pulsator), oscillation (oscillator), gyration (gyrator), or other suitable mechanism may be substituted without departing from the scope and purpose of the present invention. In some embodiments, the vibrator 380 or 480 may be remote controlled by a controller, computer, or phone app using wired or wireless (i.e. Bluetooth or other near field communication (NFC) system).

The vibrator, or other massage element, in accordance with embodiments of the invention, may impart massage to a user while the lubricant dispensing device is attached to his penis. The vibrator may also impart vibration to a female's clitoris during sexual intercourse with a male partner wearing the lubricant dispensing device. Yet further the vibrations of the vibrator may be imparted to a dildo or other device when the lubricant dispensing device is attached thereon. In such usage, the vibrator may also massage a female user's clitoris directly during usage of the massage device inside the user's vagina.

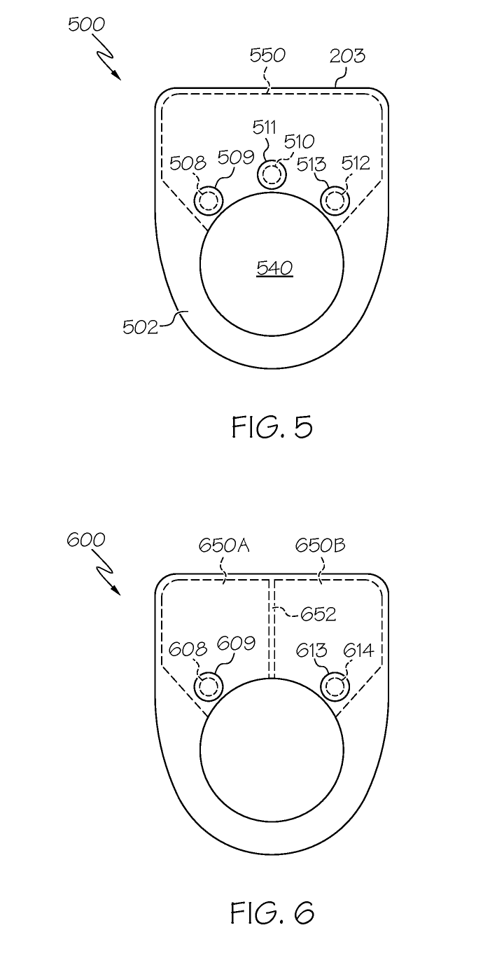

FIG. 5 shows a front view of lubricant dispensing device 500 in accordance with some embodiments of the invention. Lubricant dispensing device 500 includes three sealable openings 508, 510, and 512 positioned along the bottom of hollow chamber 550 near the upper portion of insert hole 540. Seals 509, 511, and 513 are included as well to seal openings 508, 510, and 512. In some embodiments, more than three sealable openings and seals are included. This allows for a user to dispense lubricant from the chamber in a particular area as desired.

FIG. 6 shows a front view of lubricant dispensing device 600 in accordance with some embodiments of the invention. Penis ring 600 includes two hollow chambers 650A and 650B separated by wall 652. At least two sealable openings 608 and 614 are included, one for each chamber. Seals 609 and 613 are included as well to seal openings 608 and 614, respectively. In this embodiment, two types of lubricant can be stored. For example, one having “warming” properties and one having “cooling” properties. As another example, one lubricant may be water-based and the other may be silicon-based. In some embodiments, more than two chambers are included and/or more than one sealable opening is included per chamber.

In some embodiments, the ring is an “O” ring like shown in FIG. 1. An “O” ring forms a complete and continuous circular shape. The word “circle” is used herein to mean a closed shape. The ring may be made of a resilient/flexible material, such as an elastomer. This is so that it can attach to a user's penis, or other body part or device.

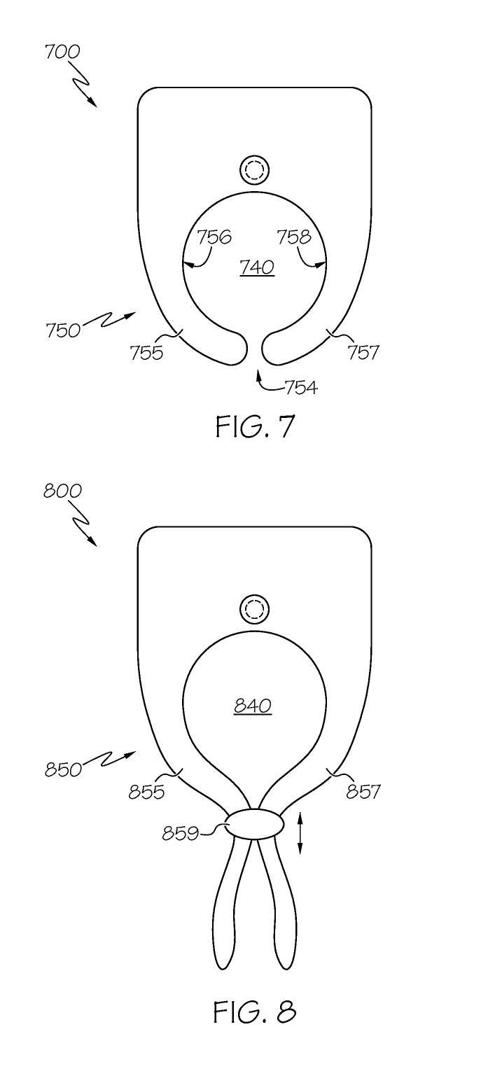

FIG. 7 shows a lubricant dispensing device 700 in accordance with some embodiments of the invention. Ring 750 is a “C” ring. A “C” ring has a discontinuity in the circle. This may mean the circle has an opening 754 through it. It may have two arms 755 and 757 having rounded sides 756 and 758 to grip the penis when inserted through insert hole 740. The two arms may be adapted to exert tension inward, i.e. toward one another. That tension holds the lubricant dispensing device on the penis when the penis is inserted through insert hole 740.

FIG. 8 shows a front view of lubricant dispensing device 800 in accordance with some embodiments of the invention. Ring 850 is formed by two string-like threads 855 and 857 and a connector 859 that clutches the threads tightly, but is still movable in an upward and downward direction. This forms a “lasso” around the user's penis, defining insert hole 840. The connector 859 can be pushed up and down to make the hole smaller or larger, respectively. This allows the user to increase or decrease the tightness of the threads around the penis. In some embodiments, a connector is not present and the threads are tied around the penis.

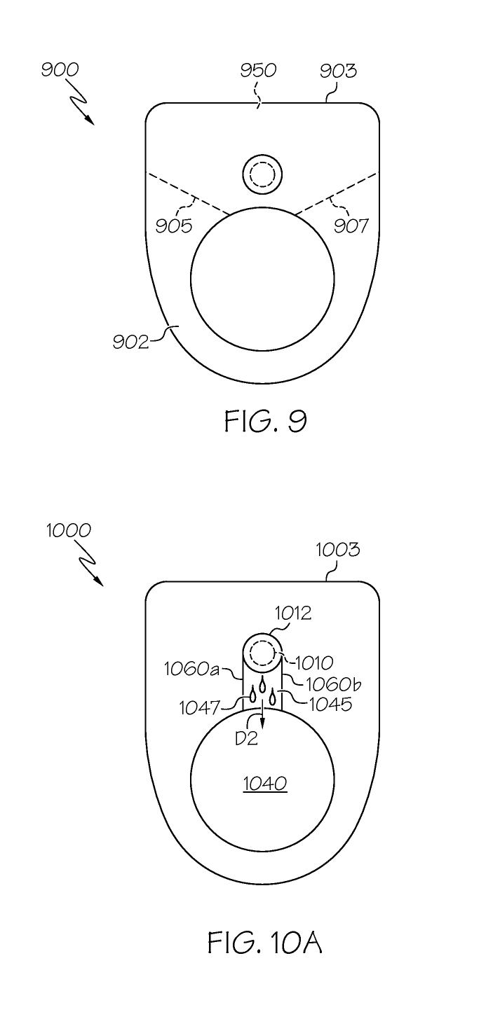

FIG. 9 shows a lubricant dispensing device 900 in accordance with some embodiments of the invention. In this example, lubricant dispensing device 900 includes a housing 903 (surrounding hollow chamber 950) formed of a firm material, such as silicon, plastic, or metal. Ring 902 is formed on its top side by the material of the housing and on its underside with an elastomeric material, such as silicon, rubber, or other suitable material. At the approximate location of chamber walls 905 and 907, the firm housing interfaces with the elastomeric ring material.

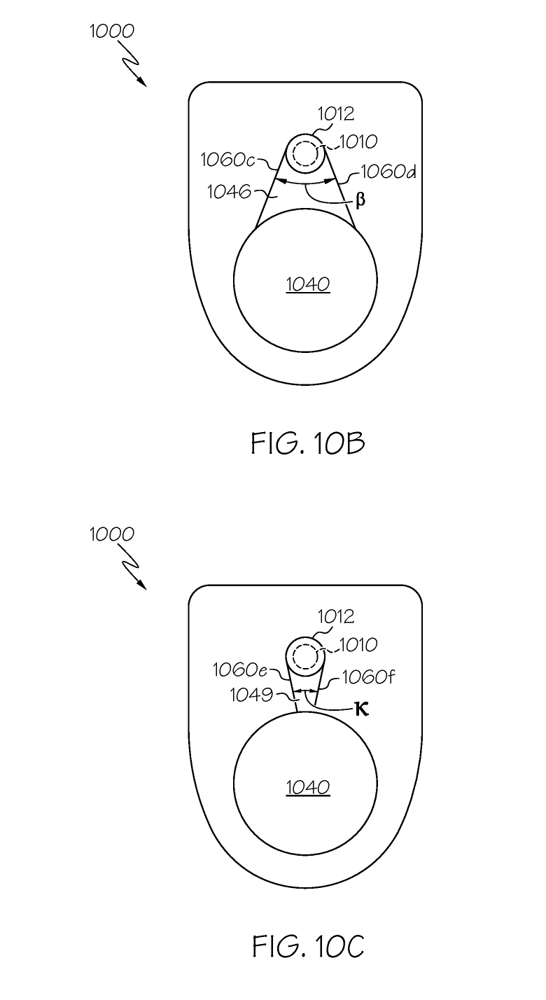

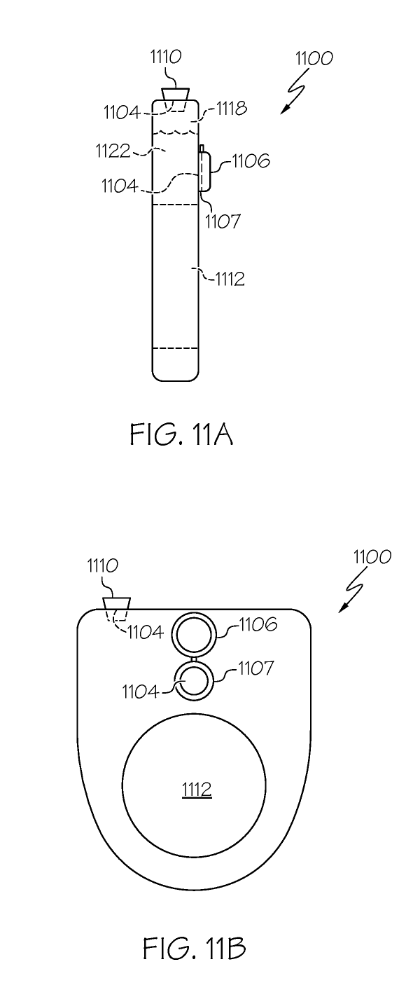

FIG. 10A shows a front view of lubricant dispensing device 1000 in accordance with some embodiments of the invention. Chamber housing 1003 includes a substantially concave depressed indentation or “channel” 1045 in to which the lubricant 1047 flows out of the hollow chamber through sealable opening 1010 (when seal 1012 is opened) and toward the insert hole 1040 (i.e. where the penis, dildo, etc. would be positioned during use) in direction D2. In some embodiments, the channel 1045 has side walls 1060a and 1060b that are substantially parallel to one another. In FIG. 10B, there is shown lubricant dispensing device 1000 with channel 1046 having side walls 1060c and 1060d that slant away from one another as they extend from the chamber to the insert hole 1040. They may slant at an angle β which in embodiments may be approximately 90 degrees or other suitable angle. In FIG. 10C, there is shown lubricant dispensing device 1000 with channel 1049 having side walls 1060e and 1060f that slant inward toward one another as they extend from the chamber to the insert hole 1040. They may slant at an angle κ, which in embodiments may be approximately 45 degrees or other suitable angle.

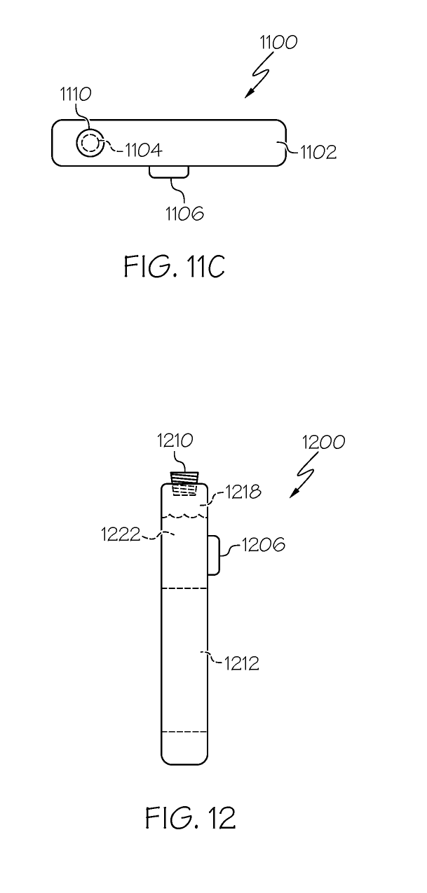

FIG. 11A-11C show views of a lubricant dispensing device 1100 in accordance with some embodiments of the invention. FIG. 11A is a side view of lubricant dispensing device 1100 with flip top cap 1106 closed. The ring includes insert hole 1112, which a penis traverses during use in attaching the ring to the penis. A cap 1110 uses a friction fit to seal opening 1104 in chamber 1118. Chamber 1118 is configured and disposed to store a lubricant 1122 or other suitable substance. The lubricant 1122 can be dispensed conveniently by removing plug 1106 briefly to allow some lubricant to exit the chamber through sealable opening 1104. FIG. 11B is a front view of lubricant dispensing device 1100 with flip top cap 1106 open. Flip top cap stays closed by a friction or interference fit of cap 1106 with opening wall 1107. FIG. 11C is a top-down view of lubricant dispensing device 1100.

FIG. 12 shows a side view of an embodiment 1200 similar to that shown in FIG. 11A, except that the cap 1210 is a threaded cap, with threads 1211 that engage with an opening in the chamber 1218. Thus, cap 1210 screws on and off to allow the chamber 1218 to be filled with lubricant 1222. FIG. 12 also shows cavity 1212 which a penis traverses during use and plug 1206.

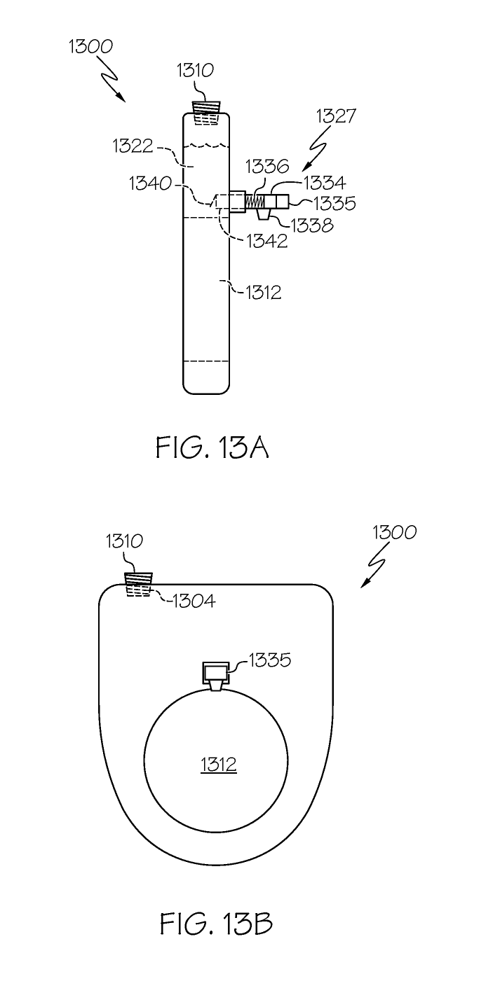

FIG. 13A shows a side view of an embodiment 1300 similar to that shown in FIG. 11A, except that the seal/sealable openings are configured differently. In embodiment 1300, instead of a plug, the first sealable opening/first seal complex is a pump dispenser 1327, used to dispense the lubricant. FIG. 13B is a front view of embodiment 1300, and FIG. 13C is a top view of embodiment 1300. A penis traverses insert hole 1312 during use. A pump head 1334 is spring-loaded by spring 1336. When the pump head 1334 is pressed towards the spring 1336, the spring 1336 compresses, and air within tube 1342 pushes one-way value 1340 away from tube 1342 to allow lubricant 1322 to enter the tube 1342. When the pump head 1334 is released, lubricant is drawn from the tube 1342 to nozzle 1338. In use, the lubricant is released from the pump head 1334, providing lubricant therefrom. In embodiments, the pump head 1334 may be configured such that when the male, wearing device 1300, is very close his partner, the partner's body may press against pad 1335 pushing pump head 1334, and causing a dispensing of lubricant. When the partners pull away from one another, the pump retracts to original position. In some embodiments, pump head 1334 has a pad 1335 thereon for comfort of the partner. Second sealable opening 1304 is configured with female threads in which male threads of seal (plus) 1310 can fit into after lubricant is poured in.

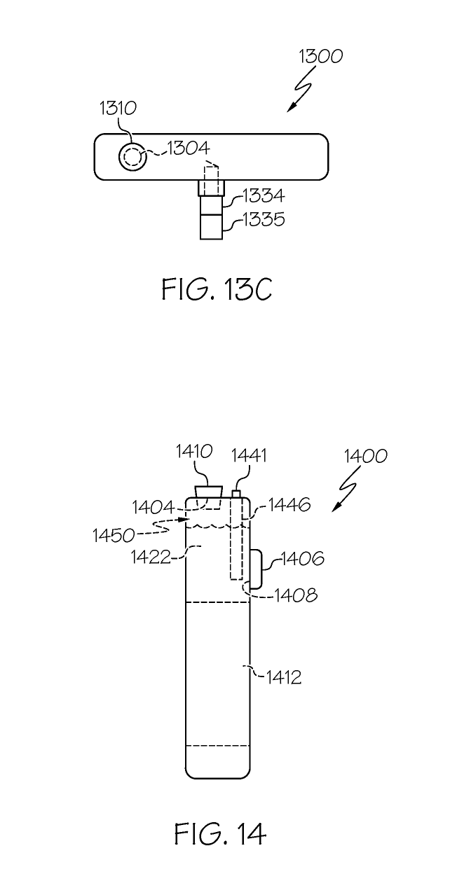

FIG. 14 shows a side view of an embodiment 1400 similar to that shown in FIG. 11A, with the addition of a heating element 1446. The heating element, at least partially within chamber 1450 can be battery operated, and can be used to elevate the temperature of the lubricant 1422, which can be dispensed by opening seal 1406 for opening 408. The heating element can be controlled via a user interface, such as a button or sensor shown generally as 1441. The heating element is in this example heats only to a preset temperature, so the button is an on/off switch. A battery and microcontroller are included as well, but not shown. A temperature sensor may also be included (not shown). The embodiment includes insert hole 1412 which a penis traverses during use, as well as second seal 1410 and second sealable opening 1404.

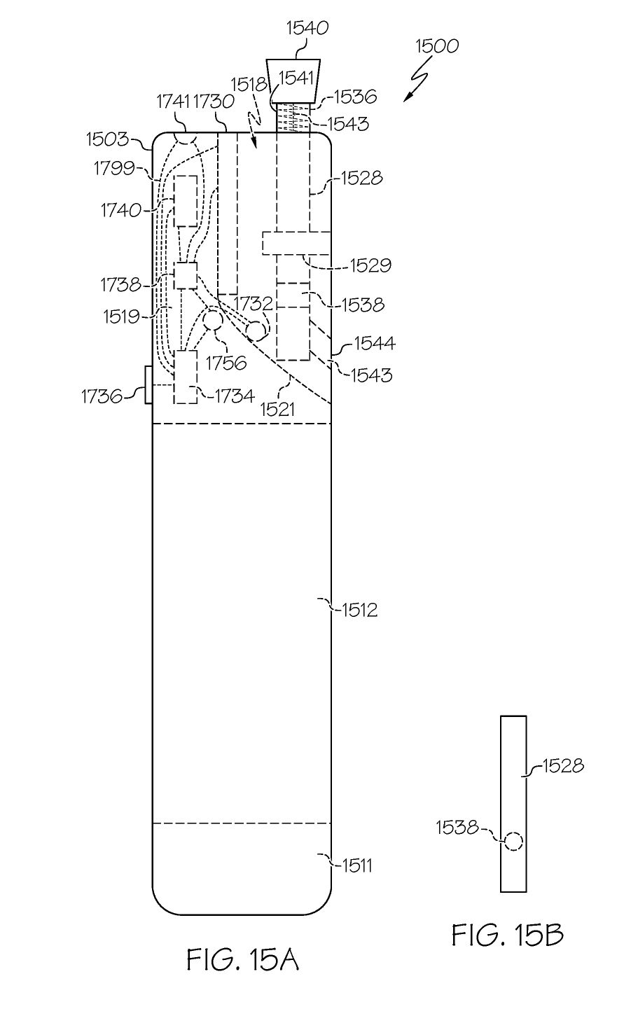

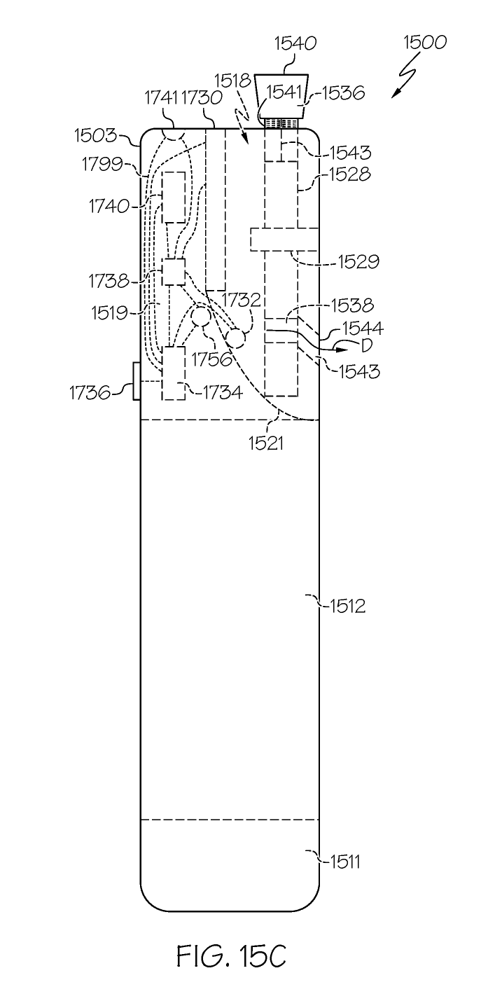

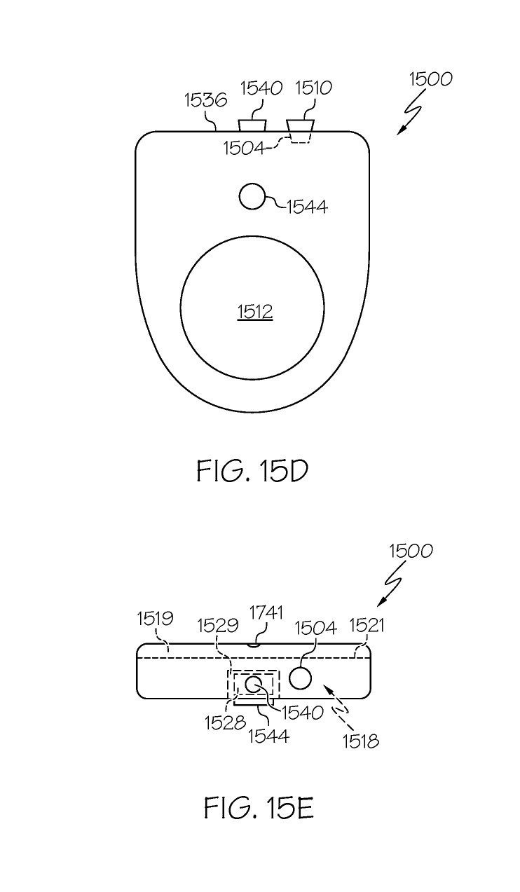

FIGS. 15A-15E show another embodiment 1500 of the present invention. FIG. 15A shows a side view of lubricant dispensing device 1500 in a non-dispensing configuration, and includes a diagram of internal electronic components (FIGS. 15B and 15D do not show the internal components in the interest of clarity). Device 1500 includes housing 1503, hollow chamber 1518, ring 1522, and insert hole 1512. In the example, the posterior and bottom wall complex 1521 of the hollow chamber 1518 forms a substantially “L” shape to guide the lubricant inside the chamber 1518 via gravity toward the direction of via 1538 and spout 1543 (i.e. the lower front/anterior wall) to opening 1544. In some embodiments, the side walls may also be curved forming a half cone-shape up to front (anterior wall). In some embodiments, at least one wall of the hollow chamber is insulated to hold in heat. Area 1519 may hold one or more of the internal electronic components. Sealable opening 1504 and seal 1510 are not shown here, but are shown in FIG. 15D.

The dispenser of lubricant dispensing device 1500 is button-operated. It includes a button 1540 which is spring-loaded by spring 1536. Button 1540 is attached to plunger 1528 by post 1543, which can be within the interior (substantial center) of spring 1536 in non-dispensing configuration. Collapsible sidewall 1541 surrounds the exterior circumference of spring 1536. Plunger 1528 has via (i.e. hole or tunnel) 1538 there through. In the configuration shown in FIG. 15A, plunger blocks (i.e. seals) spout 1543 to prevent lubricant from escaping from chamber 1518. The spout 1543 may be substantially cylindrical and substantially the same diameter as the via 1538. The spout 1543 and via may, in some embodiments, be of a shape different from a circle/cylinder. FIG. 15B shows a front view of the plunger 1528 with the via 1538. Guide 1529 guides the plunger 1528 so as to remain in close contact with the spout 1543 for dispensing or sealing. In some embodiments, further apparatuses may be included to increase the integrity of the seal created by plunger.

FIG. 15C shows a side view of lubricant dispensing device 1500 in a dispensing configuration. In operation, a user depresses button 1540, compressing spring 1536, and pushing post 1543 down with sidewall 1541 to move plunger 1528 such that via 1538 aligns with spout 1544, thereby allowing lubricant to be dispensed from chamber 1518 (i.e. flow out in direction D) through the via 1538 and spout 1543. When the user lets go of the button the spring 1536 pops back up to its non-dispensing configuration of FIG. 15A. In this way, a user can control the timing and frequency of the dispensing based on activation of button 1540. FIG. 15D shows a front view of embodiment 1500, with the addition of cap 1510 (seal) and sealable opening 1504 for receiving lubricant into the chamber 1518. FIG. 15E shows a top-down view of embodiment 1500 without cap 1510 thereon. The button 1540 is represented slightly smaller than in FIGS. 15A and 15C for clarity to show detailing of the plunger and guide 1529 thereunder.

It should be recognized that this dispenser configuration is an example, and any button-operated configuration is included within the scope of the invention. It should also be recognized that any suitable configuration of dispensing and sealing is included within the scope of the invention.

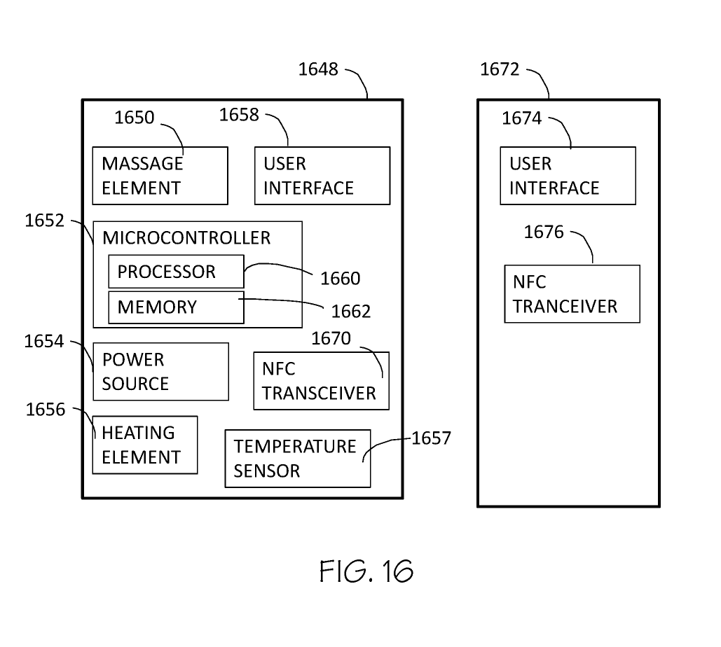

Referring again to FIG. 15A, there is shown therein a diagram of internal electronic components. Note that the term “internal” does not necessarily mean the components are within the housing. For example, a user interface may have portions that are inside as well as outside. In the example, a heating element 1730 and temperature sensor 1732 are disposed at least partially within hollow chamber 1518. Battery 1734 is in communication with charging jack 1736, temperature sensor 1732, heating element 1730, NFC transceiver 1756, microcontroller 1738, vibrator 1740, and sensor 1741. Microcontroller 1738 is in communication with battery 1734, NFC transceiver 1756, vibrator 1742, heating element 1730, temperature sensor 1732, and sensor 1741. Connections among internal components may be wired or wireless. Here, a connection is pointed out generally at 1799. Similar dashed lines indicate other connections. In embodiments, the heating element and vibrator may be remotely controlled by a remote controller such as 1672 of FIG. 16 in communication with NFC transceiver 1736. In some embodiments, the heating element and/or vibrator may be controlled by a sensor 1741, such as a pressure sensor. In some embodiments, 1741 can alternatively represent a button for controlling the vibrator and/or heating element.

It should be recognized that the embodiment of FIG. 15 is an example. The components are illustrated, but in implementations may be positioned differently within the scope of the invention. More or fewer components as feasible may be included. For example, in some embodiments, a heating element may not be included, or alternatively, a vibrator may not be included. All such configurations are within the scope of the invention. In addition, any type of dispenser is included whether button operated or operated differently.

FIG. 16 shows a block diagram 1600 of internal components 1648 of a lubricant dispensing device having a massage element and a heating element. Internal components may include a massage element 1650, a microcontroller 1652, a power source 1654, and a user interface (controls) 1658. Microcontroller 1652 may include a processor 1660 and memory 1662. The memory may include instructions 1664 for controlling the massage element 1650. In embodiments, the processor 1660 may receive input from user interface 1658. User interface 1658 may include buttons, pressure sensors, capacitive touch sensors, or other suitable user input devices. Through the user interface 1658, the user may be able to select from a plurality of vibration intensities and/or vibration patterns, as well as dispense lubricant from the hollow chamber. The processor may control massage element 1650 and heating element 1656 based on the input settings specified by the user through the user interface 1658.

In some embodiments, massage element 1650 may be a vibrator, pulsator, oscillator, gyrator, or other suitable mechanism. In some embodiments, massage element 1650 may be an electromechanical vibrator. In some embodiments, the electromechanical vibrator may be a geared motor mechanism, which may have, e.g., an asymmetrical load affixed to a rotating shaft, a linear resonant actuator, or a pancake vibration motor, etc. It will be recognized that any suitable vibration or massage mechanism now known or hereafter developed may be substituted for, or used in addition to, vibration. For example, pulsation, oscillation, gyration, or other suitable mechanism may be substituted without departing from the scope and purpose of the present invention.

In some embodiments, power source 1654 is a battery. In some embodiments, the battery may be one or more standard-sized batteries such as AA or AAA sized batteries. In some embodiments, the battery may be a permanently installed rechargeable battery chargeable through USB, inductive charging, or other suitable mechanism. The rechargeable batteries may include lithium ion batteries, nickel metal hydride batteries, or a battery of another suitable type. In some embodiments, a charging jack or electromagnetic charging system may be included in order to recharge permanently installed rechargeable batteries.

In some embodiments user interface 1658 may be a set of (at least one) button, pressure sensor, capacitive touch sensor, membrane touch switch, or other suitable interface. When the user interface 1658 is a single interface (i.e. a single sensor, button, etc.), the single interface may filter through vibration settings (i.e. high, medium, low, and patterns) as well as power on/off. For example, with use of a pressure sensor, the harder a user presses, the higher the intensity (frequency) of the vibration of a vibrator.

In some embodiments, user interface 1658 may include a membrane touch switch. In embodiments, the membrane touch switch may be configured as a momentary electrical on/off switch for activating and de-activating a circuit that provides input to the computer 809. When the membrane touch switch is pressed, its two poles make direct contact to close the circuit. When the membrane touch switch is released, the poles immediately separate and the switch returns to its original open state. In some embodiments, the membrane touch switch or button may be disposed underneath a layer of silicone, which provides a softer feel for the user.

In some embodiments, the user interface 1658 is at least one button. The button can be a momentary-on button, momentary-off button, or toggle button that toggles between an on state and an off state. In some embodiments, the user interface 1658 is at least one pressure sensor. The pressure sensor may be a piezoresistive pressure sensor, fabric potentiometer, or other suitable pressure sensor. In some embodiments, the user interface 1658 is at least one capacitive touch sensor. In embodiments, the touch sensor may include an indium tin oxide (ITO) touch sensor, printed ink touch sensor, or other suitable touch sensor technology. In some embodiments, the sensor may be a proximity sensor or a laser measuring distance of another body (or object) from the sensor.

In some embodiments, the user interface 1658 is a screen, and more particularly, a touch-sensitive screen for displaying, as well as accepting, user input.

In some embodiments, the user interface 1658 includes two interfaces, one interface (e.g., button) may be for power and the other for the heating element. In some embodiments, a touch screen and a button or sensor may be included.

In some embodiments, internal components may also include a near-field communication transceiver 1670 such as Bluetooth®, Zigbee®, or other similar device. This allows the massage element to be controlled by a paired remote controller, such as a remote control 1672.

Remote control 1672 may be a dedicated remote controller, or represent a smartphone, tablet, computer, smartwatch, or other device with a software application installed thereon. Remote control 1672 may have a near-field communication transceiver 1676, and at least one user interface 1674 (which may be at least one button and/or screen, which may be a touch-sensitive screen).

In some embodiments, the heating element 1656 can be powered on and off by user interface 1658 or 1674. In some embodiments, the user can set the desired lubricant temperature, for example, by entering a selection in the user interface 1674 or 1674, which may provide a screen with temperature option from which to choose. A temperature sensor 1657 may be included and in communication with the microcontroller 1652. When the temperature sensor senses the lubricant in the hollow chamber reaches the user-selected temperature, the microcontroller 1652 powers off (or down) the heating element. When the temperature sensor 1657 senses the temperature dips below the selected temperature, the microcontroller 1652 powers back on (or up) the heating element 1656.

For example, the user may select, via user interface 1658 or 1674, the temperature to be 85 degrees Fahrenheit (F). In the example, when the lubricant is received into the hollow chamber, it is 60 degrees F. The microcontroller powers on heating element 1656, and when the temperature sensor 1678 senses a temperature of the lubricant reaches 85 degrees F., the heating element 1656 turns off. As time goes by, if the temperature of the lubricant drops, for example to 80 degrees F., the heating element 1656 is powered back on.

In some embodiments, more than one hollow chamber may be included. In some embodiments, more than one vibrator, or other massage apparatus may be included. In some embodiments, more than one dispenser or sealable opening may be included.

The components of embodiments of the invention may be made from any suitable materials, such as plastic, elastomer, rubber, silicon, metal, glass, or any other suitable material. A non-limiting example is that the ring is made from an elastomer, and the chamber is made from plastic. In some embodiments, the housing and chamber are made from a pliable material, such as plastic, of such a density that a user can push into it to “squeeze” out lubricant out of a sealed opening if needed. Caps and other dispenser parts may be made from plastic, metal, silicon, or other suitable material. These are examples, and any suitable materials or combination thereof may be used.

It should be recognized that the dispensers and seal/sealable openings described herein are non-limiting examples. Accordingly, any suitable mechanisms for dispensing or receiving lubricant is included within the scope of the invention.

While the invention has been particularly shown and described in conjunction with exemplary embodiments, it will be appreciated that variations and modifications will occur to those skilled in the art. In particular regard to the various functions performed by the above described components (assemblies, devices, circuits, etc.), the terms used to describe such components are intended to correspond, unless otherwise indicated, to any component which performs the specified function of the described component (i.e., that is functionally equivalent), even though not structurally equivalent to the disclosed structure which performs the function in the herein illustrated exemplary embodiments of the invention. In addition, while a particular feature of the invention may have been disclosed with respect to only one of several embodiments, such feature may be combined with one or more features of the other embodiments as may be desired and advantageous for any given or particular application. For example, although some the illustrative embodiments are described herein as a series of acts or events, it will be appreciated that the present invention is not limited by the illustrated ordering of such acts or events unless specifically stated. Some acts may occur in different orders and/or concurrently with other acts or events apart from those illustrated and/or described herein, in accordance with the invention. In addition, not all illustrated steps may be required to implement a methodology in accordance with the present invention. Furthermore, the methods according to the present invention may be implemented in association with the formation and/or processing of structures illustrated and described herein as well as in association with other structures not illustrated. Therefore, it is to be understood that the appended claims are intended to cover all such modifications and changes that fall within the true spirit of the invention.

What is claimed is:

1. A lubricant dispensing device, the lubricant dispensing device comprising:
a ring;
a first hollow chamber; and
a first dispenser.

2. The lubricant dispensing device of claim 1, further comprising a vibrator.

3. The lubricant dispensing device of claim 1, further comprising a heating element.

4. The lubricant dispensing device of claim 3, further comprising a vibrator.

5. The lubricant dispensing device of claim 4, wherein the dispenser comprises a first sealable opening and a first seal.

6. The lubricant dispensing device of claim 1, wherein the first hollow chamber is configured to receive and store lubricant therein, wherein the dispenser is configured to dispense the lubricant from the first hollow chamber.

7. The lubricant dispensing device of claim 5, further comprising a second sealable opening to the chamber.

8. The lubricant dispensing device of claim 7, wherein the first sealable opening to the chamber is configured to receive lubricant into the chamber; and wherein the second sealable opening to the chamber is configured to dispense lubricant from the chamber.

9. The lubricant dispensing device of claim 3, wherein a temperature of lubricant in the chamber is user-selectable.

10. The lubricant dispensing device of claim 3, wherein the vibrator and the heating element are operated by a remote control.

11. The lubricant dispensing device of claim 4, wherein the first hollow chamber has lower walls that slant such that the lower walls increase in height as they extend from a substantially center point of the chamber floor to the side walls.

12. The lubricant dispensing device of claim 1, further comprising lubricant inside of the first hollow chamber.

13. The lubricant dispensing device of claim 4, further comprising a second hollow chamber, and a second sealable opening, wherein the first sealable opening is disposed on the first hollow chamber, and the second sealable opening is disposed on the second chamber.

14. The lubricant dispensing device of claim 4, wherein a depressed channel is disposed on a housing of the chamber extending from the sealable opening to an insert hole formed by the ring.

15. The lubricant dispensing device of claim 14, wherein the depressed channel has substantially parallel side walls.

16. The lubricant dispensing device of claim 14, wherein walls of the depressed channel angle toward one another as they extend from the dispenser to the insert hole formed by the ring.

17. The lubricant dispensing device of claim 14, wherein walls of the depressed channel angle away from one another as they extend from the sealable opening to the insert hole formed by the ring.

18. The lubricant dispensing device of claim 1, wherein the ring is formed by two threads and a connector movably bound to the two threads.

19. The lubricant dispensing device of claim 1, wherein the ring is an “O” ring.

20. The lubricant dispensing device of claim 1, wherein the ring is a “C” ring.
